(19) United States
(12) Patent Application Publication
(10) Pub. No.: US 2026/0211070 A1
(43) Pub. Date: Jul. 23, 2026

(54) ACCELERATED PROTOCOL PROCESS FOR MAGNETIC RESONANCE IMAGING

(71) Applicant: FUJIFILM Healthcare Americas Corporation, Lexington, MA (US)

(72) Inventors: Michael R. Thompson, Chesterland, OH (US); Mark Richard, Solon, OH (US)

(73) Assignee: FUJIFILM Healthcare Americas Corporation, Lexington, MA (US)

(21) Appl. No.: 19/030,593

(22) Filed: Jan. 17, 2025

(51) Int. Cl. G01R 33/56; A61B 5/055; G01R 33/48; G01R 33/561; G01R 33/565; G06T 7/00

(52) U.S. Cl. CPC G01R 33/5608; A61B 5/055; G01R 33/4818; G01R 33/5602; G01R 33/5611; G01R 33/56509; G06T 7/0012; G06T 2207/10088; G06T 2207/20081; G06T 2207/30016

(57) ABSTRACT

Provided is a method and system for accelerating magnetic resonance imaging (MRI) protocols. The method involves acquiring a foundation dataset including full k-space data, followed by acquiring target datasets with limited k-space data. The full k-space data from the foundation set is combined with the target datasets to generate high-quality images while significantly reducing acquisition time. Machine learning transformations are employed to remove artifacts associated with the data combination process. The method is particularly advantageous for low-field and low-channel count MRI systems that are less dependent on hardware limitations compared to traditional parallel imaging techniques.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright or rights whatsoever.

### TECHNICAL FIELD

The disclosed subject matter relates to the field of medical imaging and, more specifically, to methods of developing magnetic resonance imaging (MRI) protocols in medical diagnostics.

### BACKGROUND

MRI is a medical diagnostic imaging technique used to diagnose many types of medical conditions. In MRI systems, three electromagnetic fields interact to produce images of anatomy, for example, human anatomy. The three fields include:
(1) Main magnetic field—a static, spatially homogenous field to polarize spins of various nuclei within the body, making a net positive population available for detection. The static field must be very homogenous for imaging. For a horizontal field MRI system, the static magnetic field is oriented along the patient axis (head-to-foot) of a patient lying on a horizontal table. This axis is typically referred to as the z-direction.
(2) Gradient magnetic field—a spatially varying field that can create a difference in the z-component of the magnetic field across the imaging region. Additionally, the gradient magnetic field is switched at audio frequencies to encode spin positions and generate contrast. Typically, three spatially varying magnetic fields are created along the orthogonal axes to encode spins in three directions.
(3) Radiofrequency (RF) magnetic field—a magnetic field operating at tens of MHz, used to add energy to spins, detect the associated signals, and generate contrast. The direction of the RF field is orthogonal to the main magnetic field.

MRI systems can include a plurality of hardware components that work in conjunction with specialized software to produce the required magnetic fields and MRI images. MRI protocols typically involve acquiring multiple scans with different contrast to aid radiologists in making differential diagnoses. However, the time required to complete these protocols can be significant, leading to patient discomfort and reduced throughput in clinical settings.

Current methods for accelerating MRI acquisitions primarily rely on partially parallel imaging techniques. These methods synthesize k-space data by combining data collected from multiple channels of phased array receive coils. However, these techniques can be limited by the arrangement and number of receive coils and can require expensive high channel count systems to achieve significant acceleration. For MRI systems which utilize a static magnetic field oriented in the vertical direction, it is difficult to construct receive coils with many channels in which the channels are decoupled one from another.

Therefore, there remains a need for developing accelerated protocol processes that are less dependent on hardware limitations and can be applied even in low-field or single-channel MRI systems.

### SUMMARY

The purposes and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as the appended figures.

To achieve these and other advantages and in accordance with the purpose of the disclosed subject matter, as embodied and broadly described, the disclosed subject matter is directed to systems and methods for accelerating MRI protocols. For example, a method for accelerating MRI protocols is provided. The method can include acquiring a foundation dataset comprising a full set of k-space data, acquiring a target dataset comprising a subset of k-space data, and combining at least a portion of the full k-space data of the foundation dataset with the subset of k-space data of the target dataset to generate a target image. The method can also include applying a machine learning transformation to remove artifacts associated with combining the full set and the subset k-space.

As disclosed herein, the foundation dataset is one of a 3-dimensional scan or a 2-dimensional scan stack of images, and the target dataset is acquired around k=0 point. The method can include Fourier transforming the combined k-space data to image space, performing a magnitude operation, and transforming the image space back to k-space prior to applying the machine learning transformation to the target image. The method can include normalizing energies of signals from the foundation dataset and the target dataset by comparing the Nyquist energy of the signals near k=0, or by comparing the signal in shared data lines. In some embodiments, the foundation dataset and the target dataset can be acquired using parallel imaging.

As disclosed herein, a machine learning algorithm can be applied to directly transform the combined k-space to image space. In accordance with the disclosed subject matter, the foundation dataset can be acquired as the result of artificial intelligence (AI)/machine learning (ML) reconstruction, or AI/ML enhanced reconstruction, which may or may not use parallel imaging. Acquiring the target dataset can be accelerated relative to the full k-space acquisition. the foundation dataset and the target dataset can be acquired using different MRI contrast mechanisms, or with different contrast agents applied. The method can include using a single-channel coil. The method can include monitoring patient motion between the acquisition of the foundation dataset and the target dataset. The method can further comprise planning the foundation dataset, wherein planning the foundation dataset comprises automatically planning the target dataset. The method can include compensating for inter-scan motion by registering low k-space, which is k-space near k=0, data prior to combining k-space data and reconstructing images.

The disclosed subject matter further includes a non-transitory computer-readable medium storing instructions that, when executed by a processor, causes an MRI system to perform a method that can include acquiring a foundation dataset comprising full k-space data, acquiring target datasets, each comprising a subset of k-space data primarily around the k=0 point, and combining k-space data from the foundation dataset with k-space data from each target dataset to generate target images. The method can include applying a machine learning transformation to remove artifacts associated with combining the k-space datasets.

In accordance with the disclosed subject matter, a system for accelerating MRI protocols is provided. The system can include an MRI scanner, a processor, and memory-storing instructions that, when executed by the processor, cause the system to perform the aforementioned methods. The MRI scanner can utilize any number of receive channels, including only one channel. The MRI scanner can operate at any field strength.

A method for acquiring an MRI protocol is also provided. The method can include performing a localizer scan to identify the anatomy of interest, acquiring a foundation scan comprising full k-space T1-weighted data, and acquiring a plurality of contrast scans, each comprising a subset of k-space data. The method can include reconstructing images for each contrast scan using a combination of the full k-space data from the foundation scan and the subset of k-space data from each respective contrast scan. The contrast scans can include at least one of accelerated T2-weighted, T2-weighted with fat saturation, T2-weighted FLAIR, T2* scans, contrast enhanced scans, diffusion weighted scans, and angiography scans, wherein each is comprising a subset of k-space data. The foundation scan can also be the localizing scan. Acquiring the accelerated scans can include collecting primarily low k-space data around k=0 point. The method can also include applying a machine learning transformation to remove artifacts in the reconstructed images.

### BRIEF DESCRIPTION OF THE DRAWINGS

In the drawings:

FIG. 1 illustrates the concept of low k-space and high k-space.

FIG. 2 illustrates an exemplary system of acquiring the low k-space data for generating a synthesized MRI image within an MRI protocol.

FIG. 3 is a flowchart depicting the steps of an exemplary method for accelerating the MRI protocols.

FIG. 4 shows a comparison of images reconstructed using the exemplary method versus traditional full k-space acquisition.

FIG. 5 shows exemplary images reconstructed using only low-k space data.

### DETAILED DESCRIPTION

In the following description, for the purposes of explanation, numerous specific details are set forth in order to provide a thorough understanding of the present invention. It will be apparent, however, that the present invention may be practiced without these specific details. In other instances, well-known structures and devices are shown in block diagram form in order to avoid unnecessarily obscuring the present invention.

The text of this disclosure, in combination with the drawing figures, is intended to describe in prose various embodiments of the disclosed technology, at the same level of detail that is used by people of skill in the arts to which this disclosure pertains to communicate with one another. That is, the level of detail set forth in this disclosure is the same level of detail that persons of skill in the art normally use to communicate with one another to implement the inventions claimed herein.

### 1. General Overview

Reference will now be made in detail to various exemplary embodiments of the disclosed subject matter, exemplary embodiments of which are illustrated in the accompanying drawings. As used in the description and the appended claims, the singular forms, such as “a,” “an,” “the,” and singular nouns, are intended to include the plural forms as well, unless the context clearly indicates otherwise. In accordance with the disclosed subject matter, systems, and methods for accelerating MRI protocols are provided. For example, a method of accelerating MRI protocols can include acquiring a foundation dataset comprising a full set of k-space data, acquiring a target dataset comprising a subset of k-space data, and combining at least a portion of the full k-space data of the foundation dataset with the subset of k-space data of the target dataset to generate a target image. The method can also include applying a machine learning transformation to remove artifacts associated with combining the full set and the subset k-space.

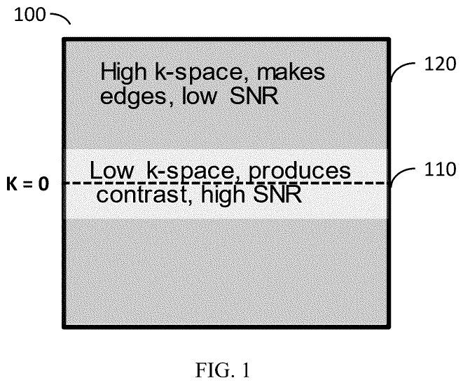

The disclosed subject matter can include systems and methods for accelerating MRI protocols by reusing common information between successive images within a protocol. For the purpose of illustration and not limitation, the fundamental structure of an MRI data acquisition in k-space is constructed with reference to FIG. 1.

The structure of a k-space is a complex 2-dimensional (2D) [or 3-D] mathematical space (100) representing the spatial frequencies of the imaged object. The center of the k-space (110), typically a small area around k=0 point, corresponds to low spatial frequencies. The center of the k-space is unique for each scan and primarily contributes to image contrast. Therefore, the center part of the k-space is unique for each scan with a different image contrast. Thus, the central region (110) in FIG. 1, corresponding to low k-space data, is acquired for each scan in the protocol.

The periphery of the k-space (120), on the other hand, corresponds to high spatial frequencies. It is more consistent across different contrast mechanisms. Therefore, data in the periphery of k-space may be more easily shared between scans which have different contrast. This data primarily contributes to image details and resolution. The periphery region (120) in FIG. 1, corresponding to high k-space data, is thus acquired in full only for the foundation scan in the protocol and then shared across all subsequent scans when synthesizing a final image. Another peculiarity of MRI scans is that the full extent of k-space is typically acquired along the readout direction with minimal scan time costs, as shown in FIG. 1.

In accordance with the disclosed subject matter, methods can include initially acquiring low k-space data (110) and high k-space data (120), then acquiring only low k-space data (110) in subsequent scans and utilizing the initial high k-space data (120) in subsequent scans.

### 2. Structural and Functional Over View

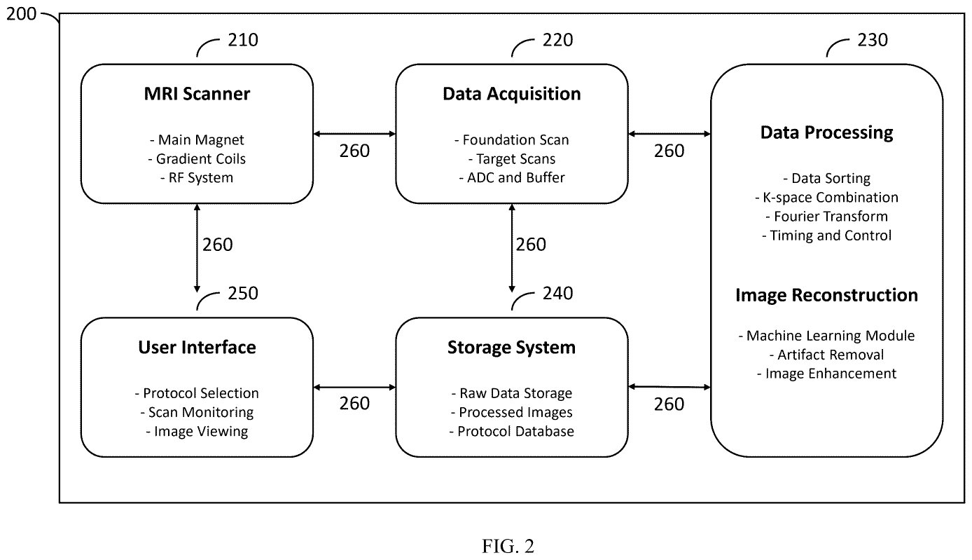

The disclosed subject matter can be implemented on a standard MRI system with modifications to the data acquisition and processing pipeline. FIG. 2 illustrates an exemplary MRI system (200) for acquiring low k-space data and generating a synthesized image within an MRI protocol. It provides a schematic overview of the hardware and software components involved in implementing the protocol. The system includes an MRI scanner (210), a data acquisition subsystem (220), a data processing subsystem (230), an image reconstruction subsystem (240), a data storage system (250), and a user interface (260).

### 2.1 MRI Scanner

The components of the MRI scanner include:
(1) Main magnet: a superconducting electromagnet configured to create a strong, uniform magnetic field (B₀) necessary for MRI. While the main magnetic field is typically 1.5T or 3.0T, both lower and higher field strengths are also used clinically and are equally applicable in the current invention.
(2) Gradient coils: coils are configured to spatially vary magnetic fields that are switched at audio frequencies to encode spin positions and generate contrast. Three orthogonal gradient coils can be used to encode spins in three directions.
(3) Radiofrequency (RF) transmit coil: a coil can be configured to generate an RF magnetic field (B₁) operating at tens of megahertz (MHz). The coil can excite spins and generate contrast.
(4) RF receive coil(s): coils can be configured to detect the MRI signal emitted by the excited nuclear spins. The system can use a single receive coil or multiple coils in a phased array configuration. The receive coils are typically constructed using copper conductors, most typically in the form of flexible printed circuit boards (PCBs).
(5) Computer system: This can include the hardware and software necessary for controlling the MRI system, data acquisition, and image reconstruction. The software will require modifications to implement the proposed acceleration method.

### 2.2 Data Acquisition Subsystem

The data acquisition subsystem (220) can collect the k-space data and can include (a) analog-to-digital converters (ADCs) and (b) data buffers. ADCs can convert the analog MRI signals detected by the receive coils into digital data. The data buffers can be a temporary storage that holds the acquired k-space data before it is transferred to the main computer system for processing.

### 2.3 Data Processing and Image Reconstruction Subsystem

The data processing and image reconstruction subsystem (230) includes a data processing unit that further includes a data sorting and combination module, a Fourier transform module, a timing and control module, and an image reconstruction unit that further includes a sophisticated machine learning module. The data sorting and combination module can organize and combine-space data collected from both foundation and target scans. Additionally, working in conjunction with this, the Fourier transform module performs the essential mathematical conversions needed to transform k-space data into image-space data. Overseeing all these operations, the timing and control module coordinates the precise timing of RF pulses, gradient switching, and data acquisition to ensure proper implementation of the desired pulse sequence. The image reconstruction unit plays a crucial role in transforming acquired k-space data into the final MRI images through several interconnected components. The sophisticated machine learning module is configured to process the combined k-space data to implement various algorithms specifically designed for artifact removal and image quality enhancement. Further refinement of the images requires this subsystem to handle all necessary post-processing steps to produce the final output.

### 2.4 Storage System

The data storage system (240) includes storage for raw data, processed images, and a protocol database. The raw data storage function can store the acquired k-space data from both foundation and target scans. The system can also store the final reconstructed images and the predefined and user-customized accelerated protocols.

### 2.5 User Interface

The user interface (250) is configured to allow the MRI technologist to interact with the system. The user interface can enable protocol selection, scan monitoring, and image viewing. The user interface can allow the user to select and configure the accelerated MRI protocol, can provide real-time feedback on the progress of the scan, and is configured to allow the user to immediately review the reconstructed images via the image viewing function.

Overall, the MRI system is configured to interconnect each component by bidirectional data flow paths (260) that facilitate the transfer of data and control signals. Primary data flow paths connect the MRI scanner (210) to the data acquisition subsystem (220), and subsequent processing subsystems. Secondary data flow paths enable communication between the data storage system (240), data processing and image reconstruction subsystem (230), and user interface (250). Additional data flow paths can connect an optional motion tracking component to the data processing and image reconstruction subsystem (230), enabling real-time motion correction during the reconstruction process.

During operation, the MRI scanner (210) acquires the foundation and target scan data under the control of the data acquisition subsystem (220). The acquired data is processed by the data processing and image reconstruction subsystem (230), which combines k-space data from the foundation and target scans, and then applies machine learning transformations to remove artifacts and enhance image quality. The processed images are stored in the data storage system (240) and can be viewed through the user interface (250).

By acquiring a full k-space dataset in an initial “foundation” scan and then combining this data with limited, primarily low k-space data from subsequent “target” scans, this integrated system enables efficient acquisition and processing of accelerated MRI data while maintaining image quality through machine learning-based reconstruction and motion correction. The system architecture allows for high and fast data flow between components while providing comprehensive monitoring and correction capabilities throughout the acquisition and reconstruction process.

### 3. Methods of Accelerating MRI Protocols

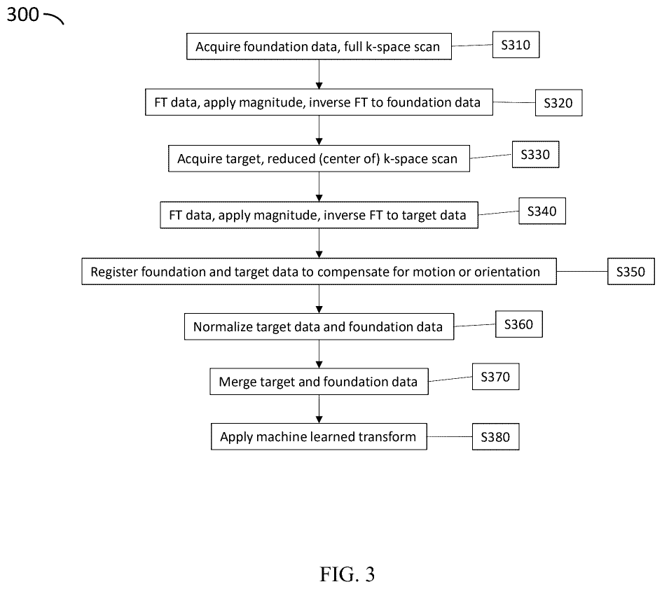

FIG. 3 illustrates a flowchart depicting the steps of the exemplary method for accelerating MRI protocols. This flowchart provides a detailed, step-by-step visualization of the process described in the Structural and Functional Overview section. The method (300) can include acquiring a foundation dataset (S310). The system can acquire a full k-space dataset for the foundation scan, which involves executing the defined pulse sequence to acquire a fast, high signal-to-noise ratio (SNR), and a full k-space dataset and storing the acquired full k-space dataset in the raw data storage. This foundation dataset can be either a 3D scan or a 2D stack of images, which can be T1-weighted and serve as the basis for subsequent accelerated scans. As another example, the foundation dataset can be T2, or PD weighted. Parallel imaging techniques can be used to accelerate the acquisition of this foundation data. In accordance with the disclosed subject matter, the foundation dataset may or may not be of clinical interest, but it should provide high-quality and high k-space data. The acquired data can undergo Fourier transformation (FT), magnitude operation, and inverse Fourier transformation (S320). Due to expected scan-to-scan and channel-to-channel variations, each MRI scan possesses phase data that mostly does not contribute to diagnosis, but makes it difficult to share data between different scans. By performing the magnitude operation in x-space and inverting the Fourier Transform, these phase issues are removed, and scan-to-scan data is made consistent.

The method can further include acquiring target datasets (S330). For each target scan in the protocol, the system can execute a modified pulse sequence to acquire only a subset of k-space data, primarily around the k=0 point. FIG. 1 shows an exemplary low k-space sampling pattern, but other center dense sampling patterns may be employed. The acquired partial k-space dataset can be stored in the raw data storage. These reduced k-space target scans can be significantly accelerated compared to the full k-space acquisition. These scans can be T2-weighted Fluid-attenuated Inversion Recovery (FLAIR). Like the foundation data, the target data can undergo FT, magnitude operation, and inverse FT (S340). Initial investigations suggest acceleration factors of up to eight may be possible. Patient motion is a practical issue in MRI scanning that causes a number of artifacts, and may render images non-diagnostic. There are many methods for monitoring and correcting motion that are known to those skilled in the art. The system can, for example, register the foundation and target data to compensate for inter-scan motion or orientation changes (S350). The MRI signal magnitude is expected to change for different MRI methods. To reduce the impact of this reality, the data can be scaled so that target and foundation data are more consistent. The target and foundation data can be further normalized to ensure consistent signal levels (S360).

The method can also include merging or combining target and foundation k-space data (S370). For each target scan, the system can first retrieve the full k-space dataset from the foundation scan. The limited k-space data collected can then be combined with the high k-space data from the foundation scan to generate the target images. The processed and normalized target and foundation data can be combined. The data sorting and combination unit can merge the two datasets, using the limited k-space data from the target scan and the high k-space data from the foundation scan data. To avoid phase problems and differences between the raw limited k-space data from different scans, all data can be Fourier transformed to image space, a magnitude operation performed, and then the data transformed back to k-space prior to combination or image formation. This step can be omitted if the target method relies on phase information. The Nyquist energy of the signal near k=0, or overlapping data lines, can be compared to scale the magnitude of the different limited k-space datasets, thus normalizing the energies of the signals from the various datasets.

The method can further include applying machine learning transformations (S380) to remove artifacts associated with combining the k-space datasets and improve image quality. These transformations can be applied either to transform the images from k-space to x-space or to improve the images after Fourier transformation. The image processing unit can perform additional post-processing steps, such as windowing and leveling. The objective of the machine learning algorithm is to combine data from a full-resolution foundation scan and a reduced k-space target scan to generate an image that is similar to a full k-space target scan. A network can be trained to transform data from k-space to x-space or x-space to x-space.

An MRI technologist can select an accelerated protocol from the user interface. The protocol can define multiple parameters: (a) foundation scan parameters include the contrast mechanism (e.g., T1-weighted) and the full k-space acquisition strategy; (b) parameters for each target scan, which include the contrast mechanisms (e.g., T2-weighted, FLAIR, etc.) and the limited k-space acquisition strategy; and (c) acceleration factors for each target scan. Upon completion, the system can display the reconstructed images on the user interface for immediate review by the MRI technologist. The images can also be stored in the processed image storage for later retrieval and analysis.

In some embodiments, a motion-tracking subsystem can continuously monitor patient motion during all scans. When significant motion is detected between the foundation and target scans, a motion correction algorithm can adjust the k-space data combination process to compensate for inter-scan motion. Where there is an absence of active motion tracking and compensation, the inter-scan motion can also be compensated by registering low k-space data prior to combining k-space data and reconstructing. Machine learning transforms may also be trained to compensate for motion between the foundation and target scans. Other motion compensation techniques used in modern MRI scanners that may be applied will be obvious to those skilled in the art.

Steps S320, S340, S350, and S360 can be optional. When dealing with phase-sensitive applications: Steps S320 and S340 (Fourier transform, magnitude operation, inverse Fourier transform) can be skipped if the phase information in the MRI signal is important for the final image contrast. The magnitude operation removes phase information, which might be necessary for certain types of MRI contrast mechanisms.

Step S350 (motion compensation) can be unnecessary if the patient remains sufficiently still between the foundation and target scans, or if the scans are acquired rapidly enough that motion is not a significant factor. When signal levels are already well-matched: Step S360 (normalization) can be skipped if the signal levels between the foundation and target datasets are inherently well-matched, which can occur when using consistent acquisition parameters.

However, for many clinical applications, one or more of these optional steps can ensure robust image quality. It is because the magnitude operations (S320 and S340) can help avoid phase-related artifacts in the combined images, the motion compensation (S350) can ensure accurate data combination even with patient movement, and normalization can ensure proper weighting when combining the datasets. Therefore, the user can consider specific clinical requirements, the type of contrast being acquired, patient compliance, hardware capabilities, and time constraints to decide whether to include or exclude these steps.

According to the disclosed subject matter, and to further demonstrate significant time savings, a comparative analysis of scan times for a typical brain MRI protocol was conducted.

Table 1 below presents the results of this analysis, comparing
conventional scan times using parallel imaging techniques with
the scan times achieved using the proposed acceleration method.

| | | | Scan Time | Scan Time |
|---|---|---|---|---|
| | | | (using parallel | (proposed) |
| | | | imaging) | (kernel16) |
| Group Task Name | Task Type | Name | [min:sec] | [min:sec] |
| *Scanogram SCA | Scan | Scano | 00:18 | 00:18 |
| Full k-space | Scan | | 1:30 | 1:30 |
| Foundation scan | | | | |
| *Axial T2 FSE | Scan | Axial T2 FSE | 01:31 | 0:10 |
| *Axial T2 FLAIR | Scan | Axial T2 FLAIR | 01:49 | 0:20 |
| *Axial T1 FSE | Scan | Axial T1 FSE | 01:39 | 0:10 |
| *Axial T2* ADAGE | Scan | Axial T2* ADAGE | 01:47 | 0:20 |
| *Axial DWI | Scan | Axial DWI | 00:46 | 0:46 |
| Total Time: | | | ~10 minutes | ~3 minutes |

In Table 1, a brain MRI protocol for a 1.5T brain protocol without contrast agent is presented for purposes of illustration, including various scan types commonly used in clinical practice. Each row represents a different scan within the protocol: Scanogram SCA is an initial localizer scan, typically used for planning subsequent scans. Its duration remains unchanged in the exemplary method. The Full k-space Foundation scan represents the acquisition of a complete k-space dataset, serving as the foundation for subsequent accelerated scans. The T2-weighted Fast Spin Echo (FSE), T2-weighted Fluid Attenuated Inversion Recovery, Axial T1-weighted FSE, and Axial T2* multi-echo gradient echo (ADAGE) represent various contrast mechanisms and orientations commonly used in brain MRI. In the illustrated conventional method, these scans can take between 1 minute 31 seconds to 1 minute 49 seconds each. Using the exemplary systems and methods, these scan times can be dramatically reduced to between 10 and 20 seconds each because only a portion of the k-space is scanned for each of these. The scan times in the example above are provided only as examples. In accordance with the disclosed subject matter, foundational scans can be collected in a few seconds or in tens of seconds. The “kernel16” notation in the exemplary method column refers to the size of the limited k-space data acquired for each accelerated scan. It shows that 16 central k-space lines are fully sampled for each contrast mechanism. Using conventional parallel imaging techniques, the entire protocol takes approximately 10 minutes. In contrast, the proposed method reduces the total scan time to approximately 3 minutes. This represents a 70% reduction in total scan time. This time saving is achieved primarily through the accelerated acquisition of the various contrast scans (Axial T2 FSE, Axial T2 FLAIR, Axial T1 FSE, and Axial T2* ADAGE). By acquiring only a small or limited portion of k-space data for these scans and combining it with the high k-space data from the foundation scan, the exemplary system and method of the disclosed subject matter can dramatically reduce the time required for each of these acquisitions while maintaining image quality.

The time savings demonstrated in this table underscore the potential to significantly improve the workflow efficiency of the MRI system. Reducing scan times from 10 minutes to 3 minutes allows for increased patient throughput, reduced patient discomfort, and improved image quality due to reduced motion artifacts. Furthermore, this acceleration can enable the addition of more advanced or complementary sequences within the same time frame as a conventional protocol, potentially enhancing diagnostic capabilities.

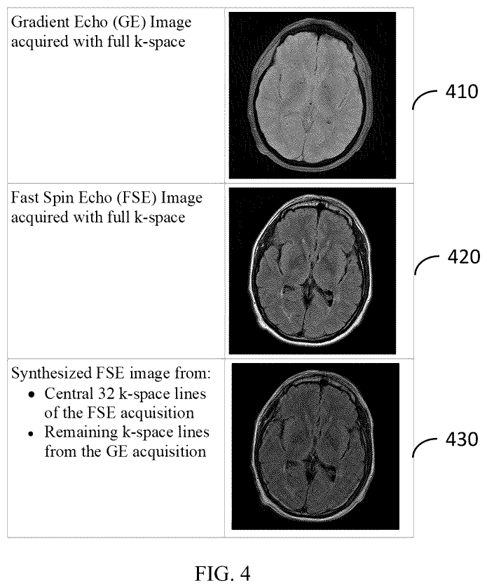

To further demonstrate the feasibility of this method, initial experiments can be performed using Fourier Transform techniques without the application of machine learning algorithms. FIG. 4 provides a visual comparison of images reconstructed using the exemplary method versus traditional full k-space acquisition. The figure includes three axial brain MR images arranged vertically, demonstrating the feasibility of the method.

The top panel (410) shows a Gradient Echo (GE) image acquired with full k-space sampling. This image represents a conventional acquisition with complete k-space coverage, serving as one of the source datasets for the synthesis experiment. The middle panel (420) shows a Fast Spin Echo (FSE) image acquired with full k-space sampling. This image represents a different contrast mechanism from the GE image, also acquired conventionally with complete k-space coverage, serving as both a reference for image quality and the source of central k-space data. The bottom panel (430) shows a synthesized FSE image created by combining data from the two acquisitions above, specifically, the central 32 k-space lines taken from the FSE acquisition (420) combined with the remaining k-space lines from the GE acquisition (410). This synthesized image demonstrates the feasibility of sharing high spatial frequency k-space data between different contrast mechanisms. While some subtle artifacts and contrast changes are visible in the bottom panel (430) compared to the fully-acquired FSE image (420), the critical anatomical features remain clearly visible and diagnostically valuable.

The comparison between the middle panel (420) and bottom panel (430) demonstrates that the synthesized image maintains much of the FSE contrast characteristics while using primarily GE acquisition data for the high spatial frequencies. This supports the fundamental premise that high spatial frequency k-space data can be effectively shared between different contrast mechanisms while maintaining diagnostic quality. The synthesized image also shows some artifacts and subtle changes in contrast to the fully acquired FSE image. However, critical features remain visible, supporting the potential of the exemplary system and method. Application of a machine learning transform can further effectively remove these artifacts and correct the contrast in the synthesized image, making it closely resemble the fully-acquired FSE image.

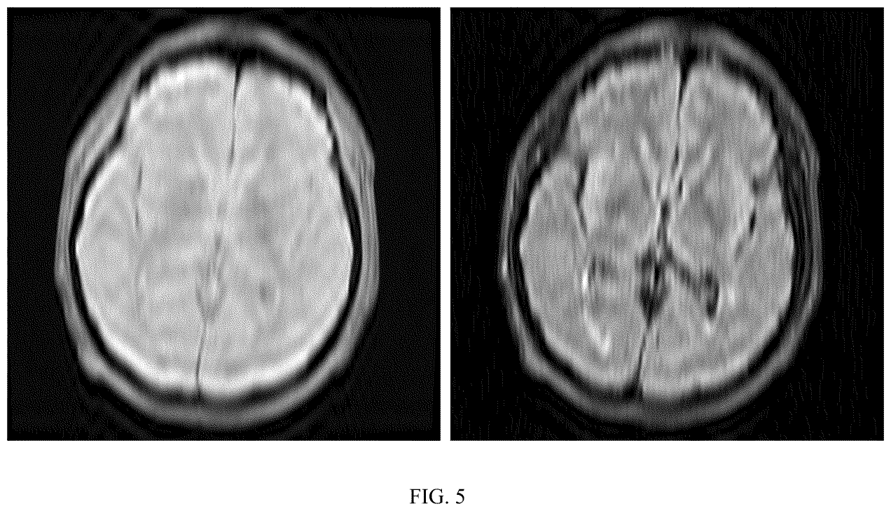

FIG. 5 demonstrates the significance of sharing high spatial frequency k-space data by illustrating brain images reconstructed from limited low k-space data only. The figure shows images reconstructed using only the low k-space lines. Specifically, image is reconstructed using the central 32 k-space lines. While the basic contrast and anatomical features are preserved in the limited k-space reconstructions, these images appear blurry and lack the fine detail visible in the full k-space acquisition (410). For example, the gray-white boundaries and small anatomical structures are increasingly less defined as fewer k-space lines are used. This visually demonstrates why sharing the high spatial frequency data from the foundation scan is crucial for it provides the detailed information that would otherwise be lost in accelerated acquisitions. In particular, the low k-space data contains the primary contrast information but requires the high spatial frequency data to achieve diagnostic quality resolution. This further illustrates the fundamental premise of the disclosed method: by acquiring only low k-space data for target scans and sharing the high spatial frequency data from the foundation scan, image quality can be still maintained while significantly reducing acquisition time.

Therefore, the exemplary method and system for accelerating MRI protocols offer several significant benefits and improvements over existing techniques.

The method provides a substantial reduction in scan time by acquiring only a small or limited subset of k-space data for target scans. The overall protocol duration can be significantly reduced. Initial investigations suggest acceleration factors of up to eight can be achieved for individual scans. It is supported by the calculated acceleration factors for some of the scans listed in Table 1. Particularly, the acceleration factors for Axial T2 FSE and Axial T1 FSE scans are 9.1 and 9.9, respectively, based on the ratio of conventional scan time to that of the exemplary method.

Despite this accelerated acquisition, image quality can be maintained by leveraging the high k-space data from the foundation scan. Furthermore, unlike parallel imaging techniques, the disclosed exemplary method does not rely on specific coil arrangements or high channel count systems. It can provide acceleration even with low channel count or single-channel coils. This hardware independence makes the method particularly cost-effective, especially for low-cost and/or low-field permanent magnet MRI systems, as it can enable faster scans without requiring expensive multi-channel coils.

The method also offers flexibility, as it can be applied to a wide range of MRI protocols and can be easily integrated into existing MRI workflows and existing MRI hardware, including various types of RF coils. For example, it can be used with breast coils, spine coils, or any other specialized coils designed for specific anatomical regions. This can be particularly beneficial for reducing the overall scan time in breast MRI protocols, which often include multiple contrast-weighted images.

Patient experience can be improved through shorter scan times, which reduce patient discomfort and the likelihood of motion artifacts. This could lead to higher-quality images and reduce the need for repeat scans. Additionally, faster protocols enable increased patient throughput, allowing more patients to be scanned in a given time period and potentially improving the efficiency and cost-effectiveness of MRI facilities.

### 4. Example Application

As a practical example of the application of the exemplary system and method of the disclosed subject matter, an accelerated protocol for comprehensive brain imaging is described as follows and shown in Table 1. First, a standard localizer scan was performed to identify the anatomy of interest, where a foundation dataset from a full k-space T1-weighted scan was acquired and served as the foundation scan for the protocol. Second, a target dataset from the accelerated target scans was acquired with limited k-space data, which included T2-weighted, T2-weighted with fat saturation, T2-weighted FLAIR, and T2*-weighted (sensitive to small differences in magnetic susceptibility between tissues).

Additionally, other contrast scan types may be employed with the accelerated acquisition method. For example, they are contrast enhanced scans that utilize injected contrast agents, diffusion weighted scans that measure the microscopic movement of water molecules in tissues, and magnetic resonance angiography scans that specifically focus on imaging blood vessels and can be performed with or without contrast agents to visualize vascular structures. Each of these contrast mechanisms can be acquired using accelerated scanning techniques, where partial k-space data is combined with the foundation scan data to produce the final reconstructed images.

Each of these target scans was acquired with a subset of k-space data, primarily around the k=0 point. The images for each scan type were then reconstructed using a combination of the full k-space data from the T1-weighted foundation scan and the subset of k-space data from each respective accelerated scan. While the disclosed subject matter is described herein in terms of certain exemplary embodiments, those skilled in the art will recognize that various modifications and improvements can be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment can be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for illustration and description purposes. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

### REFERENCE SIGNS

a. System (100)
b. Low k-space Region (110)
c. High k-space Region (120)
d. MRI Scanner (210)
e. Data Acquisition (220)
f. Data Processing and Image Reconstruction (230)
g. Data Storage System (240)
h. User Interface (250)
i. Data Flow Paths (260)
j. Accelerated Method (300)
k. Gradient Echo (GE) Image Panel (410)
l. Fast Spin Echo (FSE) Image Panel (420)
m. Synthesized FSE Image Panel (430)

1. A method for accelerating magnetic resonance imaging (MRI) protocols comprising:
acquiring a foundation dataset comprising a full set of k-space data;
acquiring a target dataset comprising a subset of k-space data; and
combining at least a portion of the full k-space data of the foundation dataset with the subset of k-space data of the target dataset to generate a target image.

2. The method of claim 1, further comprising applying a machine learning transformation to remove artifacts associated with combining the full set and the subset k-space data.

3. The method of claim 2, further comprising:
Fourier transforming the combined k-space data to image space;
performing a magnitude operation; and
transforming the image space back to k-space prior to applying the machine learning transformation to the target image.

4. The method of claim 1, wherein the foundation dataset is one of a 3-dimensional scan and a 2-dimensional scan stack of images.

5. The method of claim 1, wherein the target dataset is acquired around k=0 point.

6. The method of claim 1, further comprising:
normalizing energies of signals from the foundation dataset and the target dataset by comparing a Nyquist energy of the signals near k=0, or at other k-space locations.

7. The method of claim 1, wherein the foundation dataset is acquired using parallel imaging.

8. The method of claim 1, wherein the target dataset is acquired using parallel imaging.

9. The method of claim 1, wherein the foundation dataset is the result of artificial intelligence (AI)/machine learning (ML) reconstruction, or AI/ML enhanced reconstruction, which may or may not use parallel imaging.

10. The method of claim 1, wherein a machine learning algorithm is directly transforming the combined k-space to image space.

11. The method of claim 1, wherein the foundation dataset and the target dataset are acquired using different MRI contrast mechanisms, or with different contrast agents applied.

12. The method of claim 1, further comprising using a single channel coil.

13. The method of claim 1, further comprising planning the foundation dataset.

14. The method of claim 13, wherein planning the foundation dataset comprises automatically planning the target dataset.

15. The method of claim 1, further comprising monitoring patient motion between acquisition of the foundation dataset and the target dataset.

16. The method of claim 15, further comprising compensating for inter-scan motion by registering low k-space data prior to combining k-space data and reconstructing images.

17. The method of claim 15, further comprising compensating for motion by the machine learning transform.

18. A non-transitory computer-readable medium storing instructions that, when executed by a processor, causes an MRI system to perform a method comprising:
acquiring a foundation dataset comprising full k-space data; acquiring target datasets, each comprising a subset of k-space data primarily around a k=0 point; and
combining k-space data from the foundation dataset with k-space data from each target dataset to generate target images.

19. The method of claim 18, further comprising:
applying a machine learning transformation to remove artifacts associated with combining the k-space datasets.

20. A method for acquiring a magnetic resonance imaging (MRI) protocol, comprising:
performing a localizer scan to identify anatomy of interest;
acquiring a foundation scan comprising full k-space T1-weighted data;
acquiring a plurality of contrast scans, each comprising a subset of k-space data; and
reconstructing images for each contrast scan using a combination of the full k-space data from the foundation scan and the subset of k-space data from each respective contrast scans.

21. The method of claim 20, wherein the contrast scan comprises at least one of accelerated T2-weighted, T2-weighted with fat saturation, T2-weighted FLAIR, T2* scans, contrast enhanced scans, diffusion weighted scans, and angiography scans.
